(19) United States
(12) Patent Application Publication
(10) Pub. No.: US 2007/0224231 A1
(43) Pub. Date: Sep. 27, 2007

(54) Anti Acne Skincare Composition and Skincare Article

(75) Inventor: Timm Schmidt, Reinbek (DE)

Correspondence Address: ROTHWELL, FIGG, ERNST & MANBECK, P.C., 1425 K STREET, N.W., SUITE 800, WASHINGTON, DC, 20005, US

(73) Assignee: Reckitt & Colman (Overseas) Limited, Berkshire (GB)

(21) Appl. No.: 11/596,581

(22) PCT Filed: May 11, 2005
(86) PCT No.: PCT/GB05/01834

§ 371 Date: Apr. 11, 2007

(30) Foreign Application Priority Data
May 15, 2004 (GB) 0410860.1

(51) Int. Cl. A61K 31/60

(52) U.S. Cl. 424/402; 424/443; 514/568

(57) ABSTRACT

There is disclosed a cleansing and moisturising composition for topical application to the skin. The composition comprises salicylic acid as active ingredient, and has the form of an emulsion comprising 70-95% by weight of an aqueous phase and 5-30% by weight of an oil phase, the salicylic acid being dissolved in the oil phase, and the composition comprising one or more moisturising agents. The composition may be incorporated into a wipe substrate for ease of application to the skin. The compositions have the combined effects of cleansing and moisturising the skin and may be used in the prophylactic or remedial treatment of acne.

This invention relates to a skincare composition, and in particular to a cleansing and moisturising composition intended for topical application to the skin. The composition is particularly suitable for application by means of a wipe product, and the invention thus further provides a skincare article in the form of a substrate impregnated with, or to which is applied, the composition.

It is well-known for skincare compositions, especially those intended for cleansing of the skin, to be applied by means of wipes. Such products comprise a wipe substrate, typically in the form of a non-woven fabric, sponge, foam or other porous material, that is impregnated with the formulation. The impregnant formulations generally have a low viscosity so as to facilitate their incorporation into the wipe material.

Salicylic acid (2-hydroxy benzoic acid) is a well-known agent that is used in skincare. Salicylic acid is known in particular to be effective in the treatment of acne, and hence is used in a wide variety of cleansing products intended for the prevention or alleviation of that condition.

Wipe formulations containing salicylic acid have been marketed. Such products are water-based, but salicylic acid has only relatively low solubility in water. In order to solubilise the salicylic acid, previous products have therefore either contained high concentrations of detergent or of alcohol (eg ethanol) in order to solubilise the salicylic acid.

Unfortunately, the incorporation in the formulation of high levels of detergent or a high level of alcohol has certain disadvantages. The product may not have entirely acceptable aesthetic properties, as perceived by the user, or (where alcohol is present) may have a drying effect on the skin, or may even be irritant. The formulation may therefore be effective in cleansing the skin, but this benefit may be offset by other disadvantages. In addition, the high levels of alcohol that may be required to solubilise the salicylic acid may lead to packaging difficulties.

Alternative technologies to solubilise the salicylic acid in low viscosity compositions are known. For example, WO 0219984 discloses a wet wipe product containing an emulsion comprising relatively low amounts of salicylic acid in combination with an acrylate/C₁₀₋₃₀ alkyl acrylate cross polymer emulsifier and a non-ionic surfactant selected from the group consisting of (i) a polymeric ether, (ii) a mixture of laurate esters of sorbitol and sorbitol anhydrides condensed with ethylene oxide; and (iii) mixtures thereof. However, technical problems are encountered in providing a non-irritant low viscosity system if it is desired to increase the amount of salicylic acid and/or decrease the pH of the composition in order to improve efficacy of the salicylic acid.

There has now been devised an improved salicylic acid-containing skincare composition that overcomes or substantially mitigates the above-mentioned and/or other disadvantages associated with the prior art.

According to a first aspect of the invention, there is provided a cleansing and moisturising composition for topical application to the skin, which composition comprises salicylic acid as active ingredient, and which has the form of an emulsion comprising 70-95% by weight of an aqueous phase and 5-30% by weight of an oil phase, the salicylic acid being dissolved in the oil phase, and the composition comprising one or more moisturising agents.

The composition according to the invention is advantageous primarily in that it is effective in cleansing the skin, without the disadvantages associated with prior art formulations. In particular, the composition may include elevated amounts of salicylic acid. In addition, the pH of the composition may be sufficiently acidic to maximise the efficacy of the salicylic acid. Furthermore, as the salicylic acid is present in the oil phase of the emulsion, the formulation need not contain alcohol as it is not required to solubilise the salicylic acid in the aqueous phase. Accordingly, the undesirable drying effects of compositions containing significant amounts of alcohol are reduced. In addition, the moisturising agent present in the formulation is able to increase the level of moisture in the skin, rather than primarily overcoming the drying effect of alcohol-containing formulations. A further advantage is that the formulation is able to incorporate elevated levels of moisturising ingredients. This may contribute to a long term moisturising effect. The composition thus is able to provide a plurality of effects in that it is able to cleanse the skin, is effective in the prevention and/or treatment of acne, and moisturises at the same time. This combination of effects provides valuable therapeutic and cosmetic properties, particularly as application of the composition may achieve all these effects in a single step. The formulation also has a suitable viscosity so that it can be incorporated into wipe products. In addition, the formulation has highly desirable aesthetic properties as it is gentle on the skin, and is non-irritant. Difficulties in packaging that occur with formulations containing high concentrations of alcohol may also be avoided.

Salicylic acid is preferably incorporated into the composition according to the invention as the free acid. However, the pH of the composition may, and generally will, be such that the salicylic acid exists in the composition in dissociated form. As the composition may well contain cationic counterions, the salicylic acid may then be thought of as being present in salt form. Alternatively, the salicylic acid may be incorporated into the composition in salt form, eg as a salt with a Group I metal, such as sodium salicylate. As used herein, unless the context requires otherwise, any and all references to salicylic acid should be taken to encompass references to the acid and to dissociated forms and salts thereof. The salicylic acid may also be provided from naturally occurring sources, such as willow herb.

The concentration of salicylic acid in the composition according to the invention is preferably at least 0.1% by weight, more preferably at least 0.5% by weight. The concentration of salicylic acid is preferably less than 5% by weight, more preferably less than 4%, and most preferably less than 3% by weight. The concentration of salicylic acid may therefore fall in the range 0.1% to 5% by weight, preferably greater than 0.5% to 4%, most preferably 1% to 3% and most preferably 1-2% by weight. Particularly preferred concentrations of salicylic acid are 1%, 1.5% and 2% by weight.

As described above, the composition according to the invention is advantageous in that it need not contain substantial quantities of an alcohol, eg a lower alcohol such as ethanol or isopropyl alcohol. The composition is therefore preferably free, or substantially free, of simple aliphatic mono-alcohols such as ethanol or isopropyl alcohol. By “substantially free” is meant that the alcohol is present at a level of less than 5% by weight, more preferably less than 1% by weight, and particularly less than 0.1% by weight.

Preferably, the emulsion is in the form of a thin emulsion. This provides that the composition is of a suitable viscosity to allow it to be impregnated or absorbed into a pad made of fibrous material for application to the skin. The composition according to the invention preferably has a viscosity approximating that of water, for example less than about 100 mPa.s. It is believed that a viscosity greater than about 1,000 mPa.s would make the emulsion less suitable for use with wipe products. The viscosity may be measured using standard viscosity measuring methods, eg a Brookfield viscometer.

The oil phase of the composition according to the invention preferably constitutes between 5% and 20% w/w of the composition, more preferably between 5% and 15% w/w of the composition. The oil phase preferably comprises a blend of lipidic or hydrophibic materials. Preferably, the composition is an oil-in-water emulsion.

The oil phase of the composition preferably comprises ethers of polyalkylene glycols with aliphatic alcohols, eg polypropylene glycol alkyl ethers such as PPG-14 butyl ether, and/or PPG-15 stearil ether to solubilise the salicylic acid in the oil phase.

The oil phase of water-in-oil or oil-in-water emulsions may also comprise for example:
a) hydrocarbon oils such as paraffin or mineral oils;
b) waxes such as beeswax or paraffin wax;
c) natural oils such as sunflower oil, apricot kernel oil, shea butter or jojoba oil;
d) silicone oils such as dimethicone, cyclomethicone or cetyldimethicone;
e) fatty acid esters such as isopropyl palmitate, isopropyl myristate, dioctylmaleate, glyceryl oleate and cetostearyl isononanoate;
f) fatty alcohols such as cetyl alcohol or stearyl alcohol and mixtures thereof (eg cetearyl alcohol);
g) polypropylene glycol or polyethylene glycol ethers, eg PPG-14 butyl ether; or
h) mixtures thereof, for example, the blend of waxes available commercially under the trade name Cutina (Henkel).

The aqueous phase of the composition preferably constitutes between 80% and 95% w/w of the composition, more preferably between 85% and 95% w/w of the composition.

Preferred emulsions comprise water in an amount of at least 70% by weight, more preferably at least 75% by weight and most preferably at least 80% by weight. The upper limit of water will depend on the amounts of other ingredients incorporated in the composition so that the water may form the remainder of the composition up to 100% of the composition. A typical maximum value is less than 95% by weight, for example 90% by weight or 85% by weight.

The composition according to the invention comprises one or more moisturising agents, ie ingredients intended to increase the water content of the top layers of the skin. Examples of such ingredients are glycerin, 1,3-butylene glycol, propylene glycol, urea, panthenol, α-hydroxy acids such as lactic acid, hydrolysed proteins, hyaluronic acid, pyrrolidone carbonic acid, as well as naturally-occurring materials such as aloe barbadensis. The moisturising agents will generally be water-soluble moisturising agents.

The composition preferably comprises between 2% and 10% w/w of moisturising agents, more preferably between 4% and 8% w/w.

The composition according to the invention may also comprise one or more further topically active ingredients useful in skincare.

The pH of the composition is preferably adjusted to a value that is less than pH 5.0, more preferably less than pH 4.0. The pH is preferably greater than 2.0. The pH of the composition thus preferably lies in the range pH 2.0 to 5.0, more preferably pH 2.0 to 4.0. It is particularly preferred that the pH of the composition should be in the range pH 2.5 to 3.5, eg approximately 3.0.

The pH of the composition may be adjusted by the incorporation into the composition of one or more suitable pH-adjusting agents. Examples of pH-adjusting agents are inorganic salts such as sodium hydroxide, and organic bases such as triethanolamine. Sodium hydroxide is a particularly preferred pH-adjusting agent.

The composition according to the invention may include at least one or more emulsifiers. Most preferably, such emulsifiers include both oil phase emulsifiers and aqueous phase emulsifiers, by which is meant emulsifiers that are processed or formulated into the oil phase or aqueous phase respectively.

Many different classes of emulsifier may be suitable for inclusion in the composition according to the invention, and these will be readily apparent to those skilled in the art.

Emulsifiers used may be any emulsifiers known in the art for use in water-in-oil or oil-in-water emulsions. Known cosmetically acceptable emulsifiers include:
a) sesquioleates such as sorbitan sesquioleate, available commercially for example under the trade name Arlacel 83 (ICI), or polyglyceryl-2-sesquioleate;
b) ethoxylated esters of derivatives of natural oils such as the polyethoxylated ester of hydrogenated castor oil available commercially for example under the trade name Arlacel 989 (ICI);
c) silicone emulsifiers such as silicone polyols available commercially for example under the trade name ABIL WS08 (Th. Goldschmidt AG);
d) anionic emulsifiers such as fatty acid soaps e.g. potassium stearate and fatty acid sulphates e.g. sodium cetostearyl sulphate available commercially under the trade name Dehydag (Henkel);
e) ethoxylated fatty alcohols, for example the emulsifiers available commercially under the trade name Brij (ICI);
f) sorbitan esters, for example the emulsifiers available commercially under the trade name Span (ICI);
g) ethoxylated sorbitan esters, for example the emulsifiers available commercially under the trade name Tween (ICI);
h) ethoxylated fatty acid esters such as ethoxylated stearates, for example the emulsifiers available commercially under the trade name Myrj (ICI);
i) ethoxylated mono-, di-, and tri-glycerides, for example the emulsifiers available commercially under the trade name Labrafil (Alfa Chem.);
j) non-ionic self-emulsifying waxes, for example the wax available commercially under the trade name Polawax (Croda);
k) ethoxylated fatty acids, for example, the emulsifiers available commercially under the trade name Tefose (Alfa Chem.);
I) methylglucose esters such as polyglycerol-3 methyl glucose distearate available commercially under the name Tegocare 450 (Degussa Goldschmidt); or
m) mixtures thereof.

The emulsifiers are particularly preferably used in the form of an oil-in-water emulsion concentrate of non-ionic emulsifiers, as well as lipidic and wax-like constituents, such as that sold by Cognis under the trade name EMULGADE CM, which comprises inter alia cetearyl isononanoate, ceteareth-20, cetearyl alcohol, glyceryl stearate, glycerine, cetyl palmitate and ceteareth-12.

The composition according to the invention preferably comprises between 1% and 10% w/w of emulsifiers, more preferably less than 5% w/w. The composition thus preferably comprises a total amount of emulsifier in the range 1% to 5% w/w. The aqueous phase emulsifiers preferably account for the major proportion of the emulsifiers present. Typically, the total amount of oil-phase emulsifiers is less than 1% w/w and more preferably less than 0.5% w/w, eg in the range 0.1% to 0.5% w/w, and the total amount of aqueous-phase emulsifiers is in the range 1% to 5% w/w, more preferably 2% to 3.5% w/w. It is preferred to use a reduced amount of emulsifiers to minimise the drying effect of the composition on the skin.

The composition according to the invention may additionally comprise other components which will be well known to those skilled in the art. These include, for example:

a) Preservatives—ingredients which prevent or retard microbial growth and thus protect the composition from spoilage. Examples of preservatives include such as propylparaben, bronopol, sodium dehydroacetate, polyhexamethylenebiguanide hydrochloride, isothiazolone and diazolidinylurea.

b) Chelating agents or sequestering agents (sequestrants)—ingredients that have the ability to complex with and inactivate metallic ions in order to prevent their adverse effects on the stability or appearance of the composition. Examples of chelating agents are ethylenediamine tetraacetic acid and its salts, notably the dipotassium and especially the disodium or tetrasodium salt.

c) Perfumes and colourings.

The composition according to the invention is particularly suitable for application to the skin by means of a wipe product. Thus, according to a further aspect of the invention, there is provided a skincare article comprising a wipe substrate impregnated with, or to which is otherwise applied, a composition as described above.

The wipe substrate may have any suitable form, and such forms will be evident to those skilled in the art. Examples include non-woven sheets, foams, sponges or other porous materials. The composition may be applied to the substrate by any suitable method, which methods will also be evident to those skilled in the art. Suitable methods include, for example, dipping or immersion of the substrate in the composition, or coating of the substrate with the composition, eg by spraying. In general, the substrate should be coated with sufficient composition to achieve the desired effect when the wipe is applied to the skin, the uptake of composition in the substrate being determined by factors such as the porosity of the substrate etc. The composition should be applied as a fine film to ensure that the substrate is impregnated with a uniform composition that is dispersed evenly throughout the substrate.

Preferably, said wipe substrate is impregnated with the skincare composition in an amount in the range from 10 to 30% by weight, preferably from 15 to 25% by weight and most preferably from 18 to 22% by weight of the wipe substrate. Suitable wipe substrates comprise materials which include natural or synthetic fibres or a mixture thereof, for example cellulose and/or cotton fibres. The wipe substrate may be impregnated with the composition as a wet wipe which is arranged for immediate use to apply the skincare composition of the present invention to the skin of the user. Alternatively, the wipe substrate may be impregnated with the skincare composition and dried to form a dry wipe which requires to be wetted, for example with water, before it can be used.

In another aspect, the invention provides a method for the treatment of a person's skin, which method comprises the application to the skin of a cleansing and moisturising composition comprising salicylic acid as active ingredient, and which has the form of an emulsion comprising 70-95% by weight of an aqueous phase and 5-30% by weight of an oil phase, the salicylic acid being dissolved in the oil phase, and the composition comprising one or more moisturising agents.

It will be appreciated that the method according to this aspect of the invention may be a therapeutic method, but will often be a primarily cosmetic method, the objective of which is to reduce or eliminate externally visible, and often unsightly, symptoms of acne vulgaris.

In a further aspect, the invention provides the use of polypropylene glycol alkyl ethers to solubilise salicylic acid in a composition comprising one or more moisturising agents and being in the form of an emulsion, the emulsion comprising 70-95% by weight of an aqueous phase and 5-30% by weight of an oil phase, the salicylic acid being dissolved in the oil phase.

In a yet further aspect, the invention provides a method of preparing a cleansing and moisturising composition comprising salicylic acid as active ingredient, and which has the form of an emulsion comprising 70-95% by weight of an aqueous phase and 5-30% by weight of an oil phase, the salicylic acid being dissolved in the oil phase and the composition comprising one or more moisturising agents, comprising the steps of
(a) solubilising the salicylic acid in at least a portion of the oil phase;
(b) blending the product from step (a) with a pre-formed emulsion containing a portion of the aqueous phase;
(c) adding any further ingredients, including the moisturising agent and the remainder of the aqueous phase.

Preferably, in step (a), the salicylic acid is combined with an ether of polyalkylene glycol with an aliphatic alcohol, eg a polypropylene glycol alkyl ether, such as PPG-14 butyl ether or PPG-15 stearil ether.

Preferably, in step (b), the pre-formed emulsion is an oil-in-water emulsion concentrate. Further preferably, the pre-formed emulsion comprises a minor portion of the aqueous phase. A preferred concentrate contains all the emulsifiers contained in the composition.

Preferably, in step (c), the pH is adjusted to a pH in the range 2-4.

The invention will now be described in greater detail, by way of illustration only, with reference to the following Example.

### EXAMPLE 1

Moisturising Wipe Formulation

| Ingredient | % w/w |
|---|---|
| PPG-14 butyl ether | 8.00 |
| Glycerin | 5.45 |
| Cetearyl isononanoate | 2.25 |
| Salicylic acid | 2.00 |
| Ceteareth-20 | 1.125 |
| Cetearyl alcohol | 1.125 |
| Glyceryl stearate | 0.45 |
| Parfum | 0.20 |
| Cetyl palmitate | 0.15 |
| Ceteareth-12 | 0.15 |
| Disodium EDTA | 0.10 |
| Aloe barbadensis juice | 0.025 |
| Maltodextrin | 0.025 |
| Sodium hydroxide | 0.00012 |
| Water | to 100% |

Method of Preparation

1. 20 kg salicylic acid and 80 kg PPG-14 butyl ether (Probutyl 14; Croda) were mixed.

2. 150 kg EMULGADE CM™ (Cognis; an oil-in-water emulsion concentrate comprising 62% water, 15% cetearyl isononanoate, 7.5% ceteareth-20, 7.5% cetearyl alcohol, 3% glyceryl stearate, 3% glycerine, 1% cetyl palmitate and 1% ceteareth-12) was introduced to a mixing vessel, and the salicylic acid/PPG-14 butyl ether premix from step 1 was added.

3. Glycerin (50 kg) and Parfum (2 kg) were added.

4. The mixture was stirred for a minimum of 30 minutes, until a paste was formed.

5. The stirrer speed was reduced, and 20 kg water added.

6. The remaining water was added.

7. Mixing was continued for a minimum of 15 minutes, or until all water was added in.

8. Disodium EDTA (1 kg) and aloe Vera powder (0.5 kg; comprising 0.25 kg aloe barbadensis juice and 0.25 kg maltodextrin) were added and mixing continued for 30 minutes.

9. Sodium hydroxide was added to adjust pH to 2.8-3.2.

The composition has the form of a milky lotion, and can be used to impregnate non-woven wipes that are then packaged in a conventional manner.

1-35. (canceled)

36. A cleansing and moisturising skincare composition for impregnation into a wipe substrate arranged to cleanse the skin, prevent and/or treat acne and moisturise in a single step on topical application to the skin, which composition comprises salicylic acid as active ingredient, and which has the form of a thin emulsion having a viscosity less than 100 mPa.s, said emulsion comprising 70-95% by weight of an aqueous phase and 5-30% by weight of an oil phase, the salicylic acid being dissolved in the oil phase, and the composition comprising one or more moisturising agents.

37. A composition as claimed in claim 36, wherein the concentration of salicylic acid is in the range of greater than 0.5% to 4% by weight.

38. A composition as claimed in claim 36, wherein the concentration of salicylic acid is in the range 1% to 3% by weight.

39. A composition as claimed in claim 36, which is free, or substantially free, of ethanol.

40. A composition as claimed in claim 36, which is free, or substantially free, of aliphatic mono-alcohols.

41. A composition as claimed in claim 36, wherein the oil phase constitutes between 5% and 20% w/w of the composition.

42. A composition as claimed in claim 36, wherein the oil phase comprises a blend of lipidic or hydrophobic materials.

43. A composition as claimed in claim 36, wherein the oil phase comprises ethers of polyalkylene glycols with aliphatic alcohols.

44. A composition as claimed in claim 36, wherein the oil phase comprises polypropylene glycol alkyl ethers.

45. A composition as claimed in claim 44, wherein the oil phase comprises PPG-14 butyl ether.

46. A composition as claimed in claim 36, wherein the aqueous phase of the composition constitutes between 80% and 95% w/w of the composition.

47. A composition as claimed in claim 36, wherein the one or more moisturising agents are selected from the group consisting of glycerin, 1,3-butylene glycol, propylene glycol, urea, panthenol, α-hydroxy acids such as lactic acid, hydrolysed proteins, hyaluronic acid, pyrrolidone carbonic acid, and aloe barbadensis.

48. A composition as claimed in claim 47, which comprises glycerin.

49. A composition as claimed in claim 48, which comprises aloe barbadensis.

50. A composition as claimed in claim 36, wherein the one or more moisturising agents constitute between 2% and 10% w/w of the composition.

51. A composition as claimed in claim 36, wherein the pH lies in the range pH 2.0 to 5.0.

52. A composition as claimed in claim 36, wherein the pH lies in the range pH 2.5 to 3.5.

53. A composition as claimed in claim 36, which comprises an inorganic salt as pH-adjusting agent.

54. A composition as claimed in claim 53, wherein the pH-adjusting agent is sodium hydroxide.

55. A composition as claimed in claim 36, which includes one or more emulsifiers.

56. A composition as claimed in claim 55, wherein the one or more emulsifiers constitute between 1% and 10% w/w of the composition.

57. A composition as claimed in any claim 56, wherein the total amount of emulsifiers is in the range 1% to 5% w/w.

58. A composition as claimed in claim 36, which comprises one or more excipients selected from the group consisting of preservatives, chelating or sequestering agents, perfumes and colourings.

59. A skincare article comprising a wipe substrate impregnated with, or to which is otherwise applied, a cleansing and moisturising skincare composition in an amount of from 10% to 30% by weight of said wipe substrate, said composition being dispersed evenly throughout said substrate, which composition comprises salicylic acid as active ingredient, and which has the form of a thin emulsion having a viscosity not more than 1000 mPa.s, said emulsion comprising 70-95% by weight of an aqueous phase and 5-30% by weight of an oil phase, the salicylic acid being dissolved in the oil phase, and the composition comprising one or more moisturising agents.

60. A skincare article as claimed in claim 59, wherein the composition has a viscosity less than 100 mPa.s.

61. A skincare article as claimed in claim 59, wherein the wipe substrate is selected from the group consisting of non-woven sheets, foams, sponges and other porous materials.

62. A skincare article as claimed in claim 59 wherein the wipe substrate is impregnated with the skincare composition in an amount in the range from 15 to 25% by weight of the wipe substrate.

63. A skincare article as claimed in claim 59 wherein the substrate comprises cellulose or cotton fibres or a mixture thereof.

64. The use of polypropylene glycol alkyl ethers to solubilise salicylic acid in a cleansing and moisturizing skincare composition for impregnation evenly throughout a wipe substrate, wherein said composition comprises one or more moisturising agents and is in the form of a thin emulsion having a viscosity not more than 1000 mPa.s, said emulsion comprising 70-95% by weight of an aqueous phase and 5-30% by weight of an oil phase, the salicylic acid being dissolved in the oil phase.

65. A method for the treatment of a person's skin, which method comprises the application to the skin of a skincare article comprising a wipe substrate impregnated with a cleansing and moisturising composition that is dispersed evenly throughout the substrate, which composition comprises salicylic acid as active ingredient, and which has the form of a thin emulsion having a viscosity not more than 1000 mPa.s, said emulsion comprising 70-95% by weight of an aqueous phase and 5-30% by weight of an oil phase, the salicylic acid being dissolved in the oil phase, and the composition comprising one or more moisturising agents.

66. A method as claimed in claim 65 which method is a therapeutic method.

67. A method as claimed in claim 66 for the prophylactic or remedial treatment of acne.

68. A method as claimed in claim 65, which method is a cosmetic method.

69. A method of preparing a cleansing and moisturising composition comprising salicylic acid as active ingredient, and which has the form of a thin emulsion having a viscosity less than 100 mPa.s, said emulsion comprising 70-95% by weight of an aqueous phase and 5-30% by weight of an oil phase, the salicylic acid being dissolved in the oil phase and the composition comprising one or more moisturising agents, comprising the steps of:
(a) Solubilising the salicylic acid in at least a portion of the oil phase;
(b) Blending the product from step (a) with a pre-formed emulsion containing a portion of the aqueous phase;
(c) Adding any further ingredients, including the moisturising agent and the remainder of the aqueous phase.
